(19) United States
(12) Patent Application Publication
(10) Pub. No.: US 2012/0045416 A1
(43) Pub. Date: Feb. 23, 2012

(54) Nutritional therapy method and dietary supplement food to restore compromised, ileal lymphoid nodular hyperplasia in autistic children

(76) Inventor: Donna Louise Sullivan, (US)

(21) Appl. No.: 12/806,831

(22) Filed: Aug. 23, 2010

(51) Int. Cl. A61K 35/66

(52) U.S. Cl. 424/93.1

(57) ABSTRACT

A nutritional therapy method to restore mesolimbic compromised, ileal lymphoid nodular hyperplasia in autistic children includes: dietary restriction of casein, gluten, opiatic peptides and branched chain amino acid and the herein described nutritional therapy solution of specified composition to rebalance parasympathetic pathways over sympathatic neural responses to retain and restore digestive, immune and endocrine function, reduce metal uptake across the blood-brain barrier, reduce Ig response, and rectal and gastrointestinal inflammation and dysfunction.

### BRIEF DESCRIPTION OF THE DRAWINGS

FIG. 1 depicts a background review of human digestive system regulation;

FIG. 2a, 2b depicts an embodiment of an overall nutritional therapy process and material sourcing steps according to the present invention;

FIG. 3 depicts and embodiment of multi-stage preparation steps according to the present invention; and

FIG. 4 depicts an embodiment of process integration and process control according to the present invention.

### BACKGROUND AND DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention generally provides a nutritional method for promoting childhood neurogenesis and ameliorating autistic human ileal lymphoid nodular hyperplasia via administering food product, meal replacement, and proposed medical food composition informed by novel physiologic understanding, diet and nutrient composition and process control.

In embodiments of the present invention, the formulary includes but is not limited to shelf-stable, reconstitutable composition meeting requirements of three approved US FDA health claims (good source of protein, casein-free, gluten-free) and two structure-function health claims (i.e. contains omega-6 fish oils and DHA which have been shown to support healthy cognitive function; and contains live active probiotic cultures to promote digestive and healthy gut function).

Embodiments include nutritional therapy, including a dietary supplement, meal replacement, medical food and/or food product to restore compromised, ileal lymphoid nodular hyperplasia in autistic children and provide adequate protein nutrition. Nutritional therapy supplement delivery forms includes but is not limited to dietary powers, infusions and liquids which may be reconstituted in foods and drinks.

Persons with autism are at increased risk of compromised ileal lymphoid nodular hyperplasia and branched chain amino acid and opiatic peptide ataxia. This novel method embodies the use of specific diet and a nutrient composition designed to provide homeostasis with regard to rebalancing parasympathetic pathways over sympathatic neural responses to retain endocrine function, stimulate secretin and cholecystokinin, and promote segmentation within the small intestine to resolve ileal dysfunction associated with childhood autism.

In an embodiment of the present invention, the selection, blending, and proportionately designed ratios of specific proteins (non-peptide and non-amino acid) sources of essential amino acids, essential fatty acids, probiotic cultures, acetyl-donating groups, and buffers support duodenum pH balance, gut micro flora, provide methylation agents, ameliorate xenobiotic and environmental metal exposure, mediate cytokine and inflammatory gut response.

Patent applications have been advanced for the use of particular omega-3-phospholipids, magnesium compositions, therapeutic enzymes, carbohydrates, quercetin-containing compositions, peptide systems with morphine-precursors and novel compositions containing metals, polypeptides to address gene expression, and diagnostic autism tests using metallothionein and/or enzyme level measurements (US patent numbers: 20080070870, 20080241273 and others, 20080193436, 20080015247 and others, 20080221143, 2007025431 A1, 20040005304, 20030194719). These solutions do not address the impact of autistic mesolimbic pathway imbalances and effects of the immature endocrine and digestive system. The present invention improves efficiency of digestion and fosters normal gut microflora which aids in methylation and transsulferation of toxins independent of bloodstream metal levels, gene expression or neural development and cognition. Further, no current solution provides practical palatable, medical foods, edible meal replacements and/or dietary supplements suitable as in-home preparations or meal starters available to caregivers and guardians for autistic children aged greater than two years.

Current solutions and patent applications do not specifically address compromised neurogenesis and ileal and duodenum gastrointestinal function through daily nutritional therapy. Clinical digestive histological evidence of the use benefits of this invention are available, measurable by x-ray fluorescence and attributable to the method of preparation and designed use of product.

In an embodiment of the present invention, symbiotic nutritional therapy is used to regulate glutamate availability, reduce xenobiotic and environmental toxins through diet, ameliorate inflammation, promote digestive health, and preferentially stimulate dopaminic cognitive function as measured by standardized, international autism diagnostics.

The primary claim is made for a nutritional therapy method based upon structure-function relationships. Upon dietary restriction of casein, gluten, opiatic peptides and branched chain amino acid and the herein described nutritional therapy solution is achieved daily for at least 90 days, sympathatic division is not allowed to override parasympathetic endocrine function during digestion, glutamate cerebral levels and enterogastrone homeostasis is achieved. Adequate methylation also supports lowered (optimized) accumulation of metals in the brain, homocysteine levels, and peptide balance and turnover. Endocrine histological and biochemical biomarkers indicate normal digestive mechanisms are restored with regard to ileal and duodenum function, nitric oxide releasing neurons, including stabilized gut microflora which is vital to methylation of heavy metals such as mercury. Cerebral levels of dopamine, serotonin, glutamate and amino acids are normalized, myelin protein synthesis is restored, histamine and acetylcholine pathways achieve homeostasis, and transsulfuration optimization ensures proper metal transport. Gut segmentation mechanisms are stimulated, Ig response, and rectal and gastrointestinal inflammation is reduced.

The primary claim includes design and rationale of nutritional therapy of the novel dietary supplement and/or medical food composition of matter associated with critical nutrient ratios, nutrient delivery form and end use utility.

Upon confirmed, standardized autistic diagnosis among children of at least two years of age, dietary intervention is proposed to control sources and types of protein, which affect mesolimbic pathways and are transformed in the gut to peptides and amino acids capable of crossing the blood-brain barrier. Human histology and biochemistry is optimized to compensate for known lacks of methylation agents, enzymes and peptidases in the growth and development of autistic children. Myelin sheath proteins, neurotransmitter pathways and electrolytic balance is also affected by dietary protein quality and sources, as are the presence of xenobiotics, environmental toxins such as heavy metal exposure from radiation, pharmacology, and other forms of environmental exposure. Probiotic preparation works synergistically with optimized protein nutrition and essential fatty (omega 6 and 3) fatty acids to restore gut and hormone homeostasis and neurotransmitter pathways in autistic children at risk for essential protein malabsorption, dietary cofactor and mineral deficiency.

In an embodiment, structure-function mechanisms for particular pathways are known and approved for food dietary supplement educational marketing claims per FDA legislation. Selection of source, type, and bioactivity, order of addition, proportions and combination of necessary, essential and adequate nutrients in proper biologic form are required to establish or restore normal function in compromised individuals to manifest the biologically beneficial function.

To use an embodiment, the end use utility is important to provide accessible, affordable, portion-controlled nutrient levels for autistic children. A recommended dietary protocol is also envisioned such that daily nutrient intakes are optimized and do not compromise the claims of the patent.

In other embodiments, the design, method of production and end user utility may be classified as a foodstuff, medical food, dietary supplement, meal replacement, or proprietary trade secret component of a larger daily, weekly or time-based dietary intervention or meal plan.

Embodiments may support food product, meal replacement, proposed medical food composition and associated process control for novel method of use to restore homeostasis in autistic human ileal lymphoid nodular hyperplasia conditions. Nutritional therapy process includes 1) ability through diet and product composition and 2) process and raw material control to provide 3) end use utility and benefits to alleviate mesolimbic pathway mitigated adverse digestive disorders specifically associated with clinical diagnosis of autism spectrum disorder for children aged at least 2 years, when product invention is used for a minimum of 90 days per recommendations and with complimentary casein and gluten-free diet of adequate nutritional protein quality.

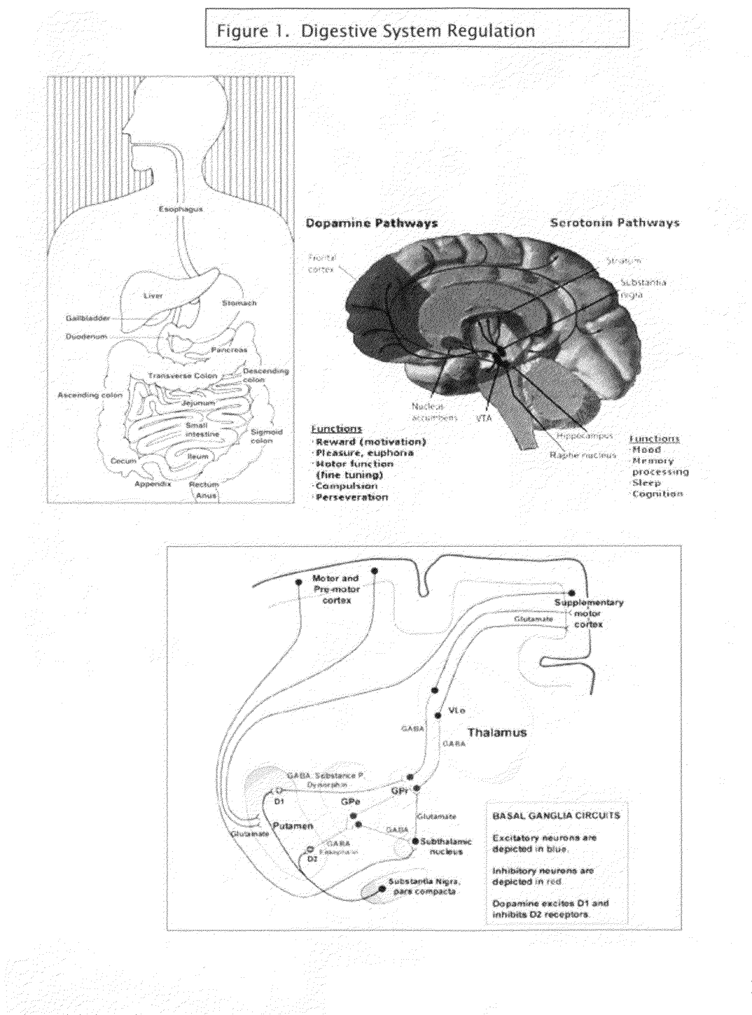

FIG. 1 illustrates digestive system anatomy, regulated by mesolimbic dopamine and serotonin pathways with details of key basal ganglia circuits.

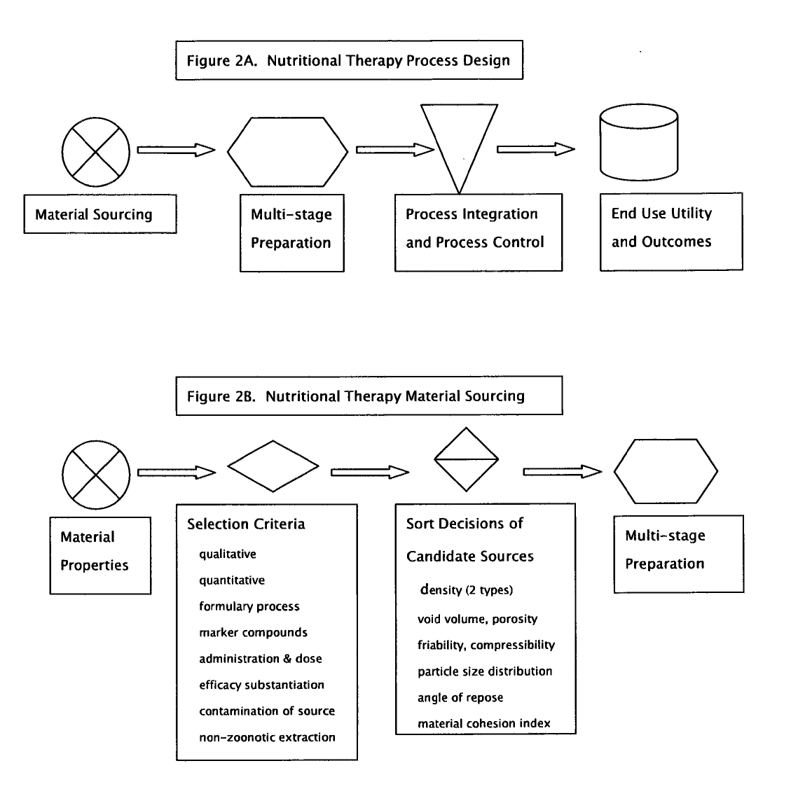

FIGS. 2a and 2b shows a nutritional therapy process/product design, process control and integration of claims to result in practical, palatable delivery forms with end user utility and benefits derived by the proposed invention.

Material sourcing step involves qualitative identity (botanical species of origin, plant part or source of extract or bioactive), descriptive, formulary extraction and solution history, quantitative potency assay via use of reference standards and accepted state of the art methods, clarification of marker compound action, pharmacology, administration and dose, and related clinical substantiation. Estimation of contamination, interferences, diluents, plating agents and solvents to include at minimum heavy metals maximum levels, IgA, G and M epitope quantification, non-zoonotic sources or at least proof of non-bovine spongiform-encephalitis source origins. Sourcing requirements include specific physical property traits related to density, friability, compressibility, cohesion, void volume and particle size distribution, and angle of repose, as these parameters affect hydraulic and pneumatic transport, bridging and arching and particle sizing and fines generation.

FIG. 2b illustrates essential product design and related process design steps related to material sourcing. Multi-stage preparation involves mixing via particular order of addition, sequence of mixing, optimized power requirements such that new surface generation is minimized. Order of addition requires minimal contact with crystallized sweeteners, buffers or mineral salts (i.e. fructose, sucrose, potassium iodine, calcium phosphate) with encapsulated, coated, or otherwise oxidation-stabilized materials until the third or later mixing step. As micronutrient levels are used, pre-mixing, sub-mixing and a minimum of three mixing stages, with each of not more than 30:1 proportion of any given material in the defined composition is required to achieve mix homogeneity. Contact surfaces shall be of stainless-steel, food contact grade material, with typical good manufacturing process (GMP) and cGMP (pharmaceutical GMP) compliance for dry-blend process. Suitable vessel designs include double-cone blenders, plow and/or ribbon blenders, and Hobart mixers, and mortar and pestle for initial mixing stages. Shear stress and energy inputs must be minimal such that the surface area: volume ratio is no greater than 1.75 over three or more stages of mixing, including final sieving/screening and canning operations.

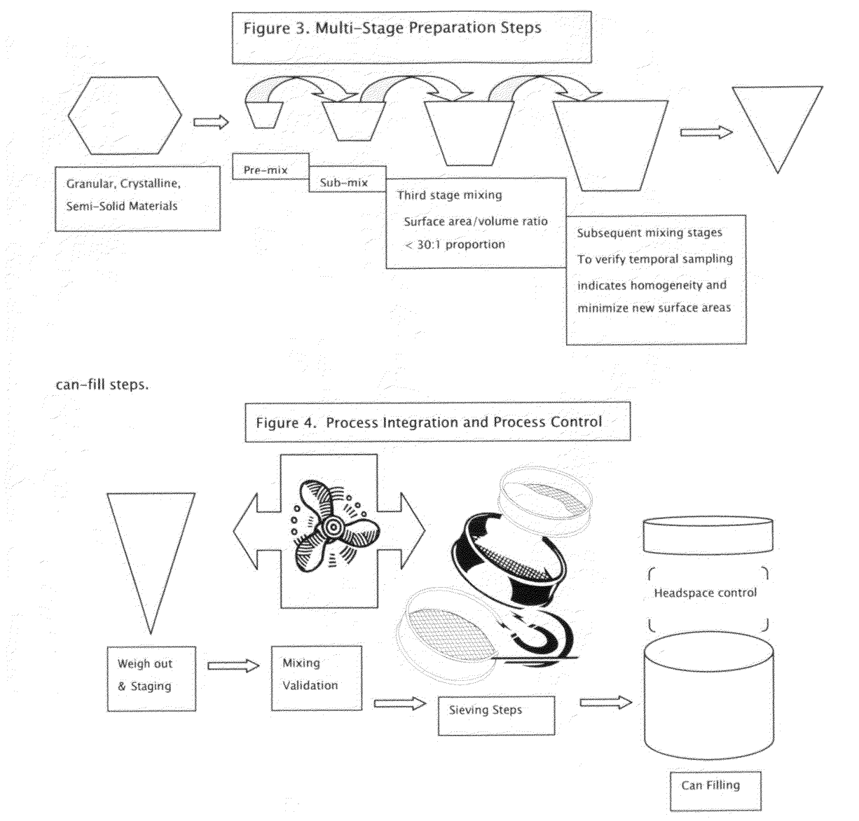

FIG. 3 illustrates the novel, innovative multi-stage preparation required for mixing the diverse component compositions of various physio-chemical properties while minimizing shear and maximizing homogeneity. Process integration and control steps are required to validate mixing efficiency, effectiveness and ensure no contamination occurred during processing, staging, and weigh-out and blending during multi-stage preparation. All stages of mixing should be conducted at constant speed with avoidance of shearing along one plane to minimize power requirements, reduce exposure of new particle surface area, avoid crystalline structure and/or encapsulated/coated particle disruption, and allow experimental estimation of adequate mix time to ensure homogeneity. Mixing time validation should be conducted through statistical sampling protocols and recalibrated after machinery, equipment or electrical disruption or off-line outage. Product sieving and metal detection process controls are recommended, per prior convention in the pharmaceutical and dietary supplement industry. Can fill and sealing procedures are directly associated with physio-chemical properties of resultant dietary supplement mix quality after multi-stage preparation, process integration and control and headspace control of oxygen level is recommended to ensure final product quality for nutritional, labeling, physical and sensory related product design parameters.

FIG. 4 illustrates critical aspects of process integration and process control for finishing and can-fill steps. End use utility and benefits of use are characterized by this novel composition, preparation, process design and control to deliver easy-to-use, home-based, non-pharmaceutical dietary supplement nutritional therapy in a shelf-stable, portion-controlled form allowing restoration of homeostasis for autistic populations with ileal lymphoid nodular hyperplasia condition.

Digestive dysfunction in clinically diagnosed autistics aged greater than 2 years can be ameliorated when product invention is used at least once daily as a dietary supplement, medical food or meal replacement as part of a dietary intervention that restricts casein and protein sources and is used continuously and ideally for a minimum of 90 days.

An embodiment of the present invention includes the following:
The nutritional therapy diet and administered product is designed to support cognitive and physical developmental nutrient requirements of children aged 2 or more years with compromised ileal lymphoid nodular hyperplasia and neurogenesis. Specifically, composition must be derived from casein and gluten-free protein (non-peptide but including essential amino acids) sources, support duodenum pH balance and gut micro flora, provide methylation agents and electrolytic homeostasis, ameliorate xenobiotic and environmental metals exposure via methylation, chelation and redox buffer control, mediate cytokine production and stimulate peptidases and sulfated glycans to support immune health.
The method of use is vital as a powdered dietary supplement reconstitutable in water, soy or rice milk or juice-containing drinks, food product and proposed medical food to support gastrointestinal function and restore homeostasis in compromised gut disorders specifically associated with clinically diagnosed autism. Autistic subjects have been shown to have increased risk of compromised gut function that is biologically distinguished from celiac disease, irritable bowel disease, Chron's disease, ulcerative colitis, opiatic peptide ataxia, peptide and epitope allergenicity and food and carbohydrate intolerance. Method of use entails dietary intervention with targeted nutritional therapy to restore and maintain duodenum and ileal function by reduction of gut permeability, promote methylation reactions and stimulation of secretin and sulfated glycosylaminoglycans in vivo production.
Process control is vital to ensure bioactivity, structure-function compliance, quality, safety, end-of-shelf-life labeling and nutritional standards and compliance with FDA and other regulatory agencies including but not limited to Orphan Drug Act compliance, and meet the regulatory requirements of dietary supplement and/or meal replacement products.

### Specifications of Nutritional Therapy Composition

Nutritional therapy ingredient specifications (both quantitative and qualitative; see FIGS. 2a and 2b) are embodied within the scope of product composition and process control to ensure intended function with regard to providing adequate but evidence-based safe levels of detoxifying nutrients, trans-methylation agents of at least 30 mg total choline per serving, electrolytic balance for intestinal homeostasis, probiotics, minimal opiatic peptide inclusion, optimal tyrosine and tryptophan to support neurotransmitter health, and maximum branched chain amino acids in formulation (not more than 0.40 g/kg body weight/day of isoleucine, leucine and valine to reduce risks of neurotransmitter dysfunction or neuropathy-associated risks associated with prolonged or extensive multiple-use on a daily basis.
End user utility is provided because of reduced risks of autism-associated conditions when used as directed for optimally 90 days, or ideally 12 months: bloated abdomen, increased or leaky gut permeability, tryptophan, tyrosine and ornithine deficiency, increased rectal inflammatory response, atoptic dermatitis, IgA, IgM and IgG allergenic response in diagnosed populations. Benefits of use include improved sleep patterns, extended eye contact, mediation of cytokine production, optimized neurotransmitter function with regard to histamine, serotonin, acetylcholine and dopamine pathways, improved attention, concentration and focus as measured by international disease classification construct rating mechanisms and validated by World Health Organization International Classification of Diseases, American Psychological Association, Lord's Autism Diagnostic Inventory and Observation Schedules and Gilliam Autism Scaling systems.

The administration of novel nutritional therapy principles within an embodiment of this invention is addressed by a dietary recommendations and formulary with specific process control and dietary compliance.

In an embodiment, the sources, purity, bioactivity, proportionate ratios of amino acid, minerals, cofactors, nutrients, electrolytes, vitamins, pH and buffer agents and formulation parameters are required in an integrated, symbiotic manner to achieve mesolimbic pathway balance and distinct support of ileal nodular hyperplasia when used as directed for at least 90 days.

An embodiment of this product can be marketed and distributed as a dietary supplement, medical food or meal replacement product under US FDA regulations and global legislation.

An embodiment includes a method for promoting childhood neurogenesis and ameliorating human autistic ileal lymphoid nodular hyperplasia via administration of specific food product, meal replacement, proposed medical food composition informed by novel and non-obvious process control: including but not limited to the use of casein and gluten-free protein sources (e.g. soy, egg, whey) containing eight essential amino acids, controlled maximum level of opiatic peptides and total branched chain amino acids of not more than 0.4 grams/kg body weight, probiotic active live cultures of at least 3 million units/serving, essential fatty acids including a weight ratio of omega-6:omega-3 oils of less than 15:1 by weight, DHA level of at least 0.2 g/serving, and total omega 3 and 6 oils to alpha lipoic acid ratio of not less than 2:1 ratio by weight, total folate and choline fortification of at least 30 mg/serving as methylation agents, potassium iodine electrolytes and buffers, water soluble B vitamins and C vitamins at one-third recommended daily level for children age 2-12 years, acetyl-donating nutrient sources of at least 0.40 g/serving, and mineral-based cofactors including phosphatidyl nutrient sources not to exceed 0.5 grams/serving.

An embodiment may include:
Novel process and material control to ensure proper bioactivity, structure-function mechanism, end-of-shelf-life-labeling compliance and caloric requirements to achieve meal replacement guidelines while controlling protein and nutritional therapy total composition of matter requirements to restore autism-related ileal site digestive dysfunction.
End use utility and benefits alleviate adverse events and stimulate healthy gastrointestinal function optimally 90 days of continued use per requirements and as part of a controlled diet menu plan to retain dietary benefits of casein and gluten-free protein consumption.

An embodiment includes the method for promoting childhood neurogenesis and ameliorating human autistic ileal lymphoid nodular hyperplasia via administration of specific food product, meal replacement, proposed medical food composition informed by novel and non-obvious process control: including but not limited to the use of casein and gluten-free protein sources and formulary included by not limited to shelf-stable food composition meeting requirements of three approved US FDA educational health claims (good source of protein, casein-free, gluten-free) and two structure-function health claims (i.e. contains omega-6 fish oils and DHA which have been shown to support healthy cognitive function; and contains live active probiotic cultures to promote digestive and healthy gut function informed through novel and non-obvious process and raw material control to provide end use utility and benefits to alleviate adverse digestive disorders specifically associated with clinical diagnosis of autism spectrum disorder for children aged at least 2 years, when product invention is used for a minimum of 90 days per recommendations and with complimentary casein and gluten-free diet of adequate protein.

It should be understood, of course, that the foregoing relates to exemplary embodiments of the invention and that modifications may be made without departing from the spirit and scope of the invention as set forth in the following claims.

We claim:

1. A nutritional therapy method for restoring mesolimbic pathway compromised, ileal lymphoid nodular hyperplasia in autistic children comprising:
dietary restriction of casein, gluten, opiatic peptides and branched chain amino acid and the herein described nutritional therapy solution is achieved for at least 90 days; rebalancing parasympathetic pathways over sympathatic neural responses to retain endocrine function, restoring histological and biochemical mechanisms with regard to stimulation of secretin and cholecystokinin, and promote segmentation within the small intestine ileal and duodenum functions, normalizing cerebral levels of glutamate and amino acids, reducing metal uptake across the blood: brain barrier, restoring myelin protein synthesis, reducing histamine and homocysteine mediated imbalance, optimizing serotonin acetylcholine and dopamine pathway homeostasis, reducing Ig response, and rectal and gastrointestinal inflammation.
